(19) United States
(12) Patent Application Publication
(10) Pub. No.: US 2011/0091873 A1
(43) Pub. Date: Apr. 21, 2011

(54) INTEGRATED SAMPLE PREPARATION AND AMPLIFICATION FOR NUCLEIC ACID DETECTION FROM BIOLOGICAL SAMPLES

(75) Inventor: Shanavaz L. Nasarabadi, Livermore, CA (US)

(73) Assignee: MicroFluidic Systems, Inc., Fremont, CA (US)

(21) Appl. No.: 12/603,428

(22) Filed: Oct. 21, 2009

(51) Int. Cl. C12Q 1/68; C12P 19/34; C12M 1/00; C12M 1/34

(52) U.S. Cl. 435/6; 435/91.2; 435/289.1; 435/287.2

(57) ABSTRACT

Beads are used to perform both a sample preparation and amplification process within a single processing chamber. A fluid sample including a plurality of cells is introduced into a processing chamber already including a plurality of beads. The plurality of cells are lysed within the processing chamber, and nucleic acid released from the lysed cells binds to the beads. The beads are retained in the processing chamber while the lysing reagents are removed. Amplification reagents, such as PCR reagents are added to the processing chamber and an amplification process is performed on the contents of the processing chamber. Nucleic acid captured on the beads is not eluted for amplification, instead amplification of the captured nucleic acid is performed while the nucleic acid is still bound to the beads. During the amplification process, the nucleic acid bound to the beads is amplified. Real-time or end-point detection can be performed on the content of the processing chamber to detect the presence of amplified product.

### GOVERNMENT LICENSE RIGHTS

This invention was made with Government support under Agreement No. W81XWH-04-9-0010 awarded by the Government. The Government has certain rights in this invention.

### FIELD OF THE INVENTION

The invention relates to a method of and apparatus for performing sample preparation and amplification for nucleic acid detection. More particularly, the invention relates to a method of and apparatus for integrating sample preparation and amplification for nucleic acid detection from biological samples.

### BACKGROUND OF THE INVENTION

Polymerase chain reaction (PCR) is a molecular technique for enzymatically replicating specific DNA sequences. In particular, PCR is used to amplify relatively short, well-defined nucleotide sequences within a given DNA strand. A specific DNA sequence to be amplified is determined by selecting primers. Primers are short, artificial DNA strands, often not more than fifty and usually only 18 to 25 base pairs long that are complementary to the beginning and end of the specific DNA sequence to be amplified. The primers bond to the DNA strand at these starting and ending points and begin the synthesis of the new DNA strand.

Analytes, such as nucleic acids from a target organism, are typically part of a larger sample, with the rest of the material within the sample ranging from trace amounts to very abundant. These materials often interfere with or completely prevent detection of the organism and can make quantitative results impossible. For example, in PCR amplification, the Taqman® polymerase enzyme is vulnerable to inhibitors present in samples. Thus, most nucleic acid detection methods require some extent of sample preparation of the material to be amplified and analyzed. Various extraction protocols and devices have been used to purify the sample, most of which are optimized for certain samples and applications, and usually require bench-top equipment used within a laboratory environment by highly skilled personal.

Most sample preparation methods involve some method of preparing nucleic acid. This is most often a two step process. First, the nucleic acid is isolated. Second, the isolated nucleic acid is amplified. There are many processes for preparing a sample. An exemplary processing technique uses silica based binding of nucleic acid followed by release of the nucleic acid using heat and a slightly alkaline pH buffer compatible with PCR. A sample is first lysed to release the nucleic acid. The lysate including the nucleic acid is then introduced to silica beads. Nucleic acid is known to bind with silica beads. The remaining lysate is removed, leaving the beads with the captured nucleic acid. The captured nucleic acid is eluted from the beads using an elution buffer. The eluate is then moved to a thermal cycling chamber for amplification of the eluted nucleic acid.

The sample preparation process is not 100% efficient and usually the sample is eluted in a volume several fold more than that used for the subsequent amplification step. For samples with a limited amount of nucleic acid, or if the process necessitates sensitivity, then the use of even a small amount of sample preparation material may be deleterious to the process. In a high through-put format, it becomes advantageous to have as few a steps as possible to mitigate losses and failure points in the process.

### SUMMARY OF THE INVENTION

An integrated apparatus and method combines both the sample preparation and the amplification process of nucleic acid on beads within a single processing chamber. A fluid sample including a plurality of cells is introduced into a processing chamber already including a plurality of beads. The plurality of cells are lysed within the processing chamber, and nucleic acid released from the lysed cells binds to the beads. The beads are retained in the processing chamber while the lysing reagents are removed. Amplification reagents, such as PCR reagents are added to the processing chamber and an amplification process is performed on the contents of the processing chamber. Nucleic acid captured on the beads is not eluted for amplification, instead amplification of the captured nucleic acid is performed while the nucleic acid is still bound to the beads. During the amplification process, the nucleic acid bound to the beads is amplified. Real-time or end-point detection can be performed on the content of the processing chamber to detect the presence of amplified product. Sample preparation on paramagnetic or silica beads followed by amplification of the nucleic acid signature on the same media integrates the sample preparation, amplification, and analysis methods within a single processing chamber, from start to finish.

In one aspect, a method of processing a sample is disclosed. The method includes providing a processing vessel including a plurality of beads, each bead is configured to bind with nucleic acid; adding a fluid sample and lysing reagents to the processing vessel, the fluid sample including one or more different types of cells; lysing the cells within the processing chamber, thereby releasing nucleic acid from within the cells, wherein the nucleic acid binds to the plurality of beads; retaining the plurality of beads within the processing chamber while removing the fluid sample and lysing reagents; adding amplification reagents to the processing vessel; and performing an amplification process within the processing vessel, wherein the nucleic acid bound to the plurality of beads is amplified. In some embodiments, performing the amplification process yields an amplified product including the nucleic acid bound to the beads and nucleic acid in solution. The method can also include detecting the amplified nucleic acid within the processing vessel. The amplified nucleic acid can be detected in real-time or at the end of the amplification process. The amplification process can be polymerase chain reaction. Lysing the cells can include sonicating the cells, applying heat to the processing vessel, or both sonicating the cells and applying heat to the processing vessel. In some embodiments, each bead is a paramagnetic bead, and retaining the plurality of beads comprises applying a magnet to the processing vessel. The method can also include adding a wash buffer to the processing chamber after removing the fluid sample and lysing reagents, and retaining the plurality of beads within the processing chamber while removing the wash buffer. In some embodiments, each bead is a silica bead, and retaining the plurality of beads includes filtering the beads as the fluid sample and lysing reagents are removed from the processing vessel. In other embodiments, each bead is coated with one or more capture moieties, each capture moiety configured to bind with a specific type of nucleic acid, and released nucleic acid of the specific types binds to the plurality of beads.

In another aspect, an apparatus for processing a sample is disclosed. The apparatus includes a sample input chamber configured to receive as input a sample including a plurality of cells; and a processing chamber including a plurality of beads positioned therein, each bead is configured to bind with nucleic acid, wherein the processing chamber is coupled to the sample input chamber to receive a fluid sample that includes the plurality of cells, further wherein the processing chamber is configured to receive lysing reagents and to lyse the plurality of cells, thereby releasing nucleic acid that binds to the plurality of beads, to remove the fluid sample and lysing reagents while retaining the plurality of beads, to receive amplification reagents, and to perform an amplification process thereby amplifying the nucleic acid bound to the plurality of beads. In some embodiments, at least a portion of the processing chamber is transparent or provides optical access to the interior to detect the amplified nucleic acid. In some embodiments, the processing chamber includes a mounting seat configured to be removably coupled to a sonication device, further wherein the processing chamber and the sonication device are configured to lyse the plurality of cells using sonication. In some embodiments, the processing chamber includes a first surface configured to be removably coupled to a heat exchanger, further wherein the processing chamber and the heat exchanger are configured to thermally cycle contents of the processing chamber, the thermal cycling used to perform the amplification process. The processing chamber and the heat exchanger can be further configured to lyse the plurality of cells by heating the contents of the processing chamber. In some embodiments, the processing chamber includes a second surface configured to be removably coupled to a magnet, further wherein each of the beads is a paramagnetic bead, and the processing chamber and the magnet are configured to retain the plurality of beads within the processing chamber while the fluid sample and lysing reagents are removed. In other embodiments, the processing chamber includes a filter coupled to each fluid input port and to each fluid output port to retain the plurality of beads within the processing chamber while the fluid sample and lysing reagents are removed. The apparatus can also include one or more solutions modules coupled to the processing chamber to provide the lysing reagents and the amplification reagents. In some embodiments, each bead is coated with one or more capture moieties, each capture moiety configured to bind with a specific type of nucleic acid, further wherein released nucleic acid of the specific types binds to the plurality of beads. The apparatus can also include microfluidic circuitry coupled to the sample input chamber and to the processing chamber, the microfluidic circuitry configured to regulate fluid flow between the sample input chamber and the processing chamber, and to regulate fluid flow into and out of the processing chamber. In some embodiments, the apparatus forms an integrated cartridge.

In yet another aspect, a system for processing a sample is disclosed. The system includes a sample input chamber configured to receive as input a sample including a plurality of cells; a processing chamber including a plurality of beads positioned therein, each bead is configured to bind with nucleic acid, wherein the processing chamber is coupled to the sample input chamber to receive a fluid sample that includes the plurality of cells; lysing means coupled to the processing chamber to lyse the plurality of cells within the processing chamber, thereby releasing nucleic acid that binds to the plurality of beads; retention means coupled to the processing chamber to retain the plurality of beads within the processing chamber while fluid is removed from the processing chamber; and amplification means coupled to the processing chamber to amplify the nucleic acid bound to the plurality of beads. The lysing means can include a sonication device. The lysing means and the amplification means can include a heat exchanger. The system can also include microfluidic circuitry coupled to the sample input chamber and to the processing chamber, the microfluidic circuitry configured to regulate fluid flow between the sample input chamber and the processing chamber, and to regulate fluid flow into and out of the processing chamber. The system can also include one or more solutions modules coupled to the processing chamber to provide lysing reagents, amplification reagents, and buffer solutions. In some embodiments, each bead is coated with one or more capture moieties, each capture moiety configured to bind with a specific type of nucleic acid, further wherein released nucleic acid of the specific types binds to the plurality of beads. In some embodiments, the retention means includes a filter coupled to each fluid input port and to each fluid output port of the processing chamber, the filters are configured to retain the plurality of beads within the processing chamber while fluid removed from the processing chamber. In other embodiments, the retention means includes a magnet configured to be removably coupled to the processing chamber, further wherein each bead is a paramagnetic bead, and the processing chamber and the magnet are configured to retain the plurality of beads within the processing chamber while fluid removed from the processing chamber.

### BRIEF DESCRIPTION OF THE DRAWINGS

The accompanying drawings, which are incorporated in and form a part of this specification, illustrate embodiments of the integrated apparatus and method and, together with the description, serve to explain the principles of the integrated apparatus and method but not limit the integrated apparatus and method to the disclosed examples.

FIG. 1 illustrates an exemplary block diagram of an integrated device according to an embodiment.

FIG. 2 illustrates a side view of processing chamber of FIG. 1.

FIG. 3 illustrates a flow chart of the sample processing method according to an embodiment.

FIG. 4 illustrates an exemplary graph of the amplification versus number of cycles for the sample preparation and amplification process applied to a Bacillus anthracis nucleic acid.

FIG. 5 illustrates an exemplary gel electrophoresis analysis performed on an amplified Human Pappiloma Virus (HPV).

Embodiments of the integrated apparatus and method are described relative to the several views of the drawings. Where appropriate and only where identical elements are disclosed and shown in more than one drawing, the same reference numeral will be used to represent such identical elements.

### DETAILED DESCRIPTION OF THE PRESENT INVENTION

Reference will now be made in detail to the embodiments of the integrated apparatus and method, examples of which are illustrated in the accompanying drawings. While the integrated apparatus and method will be described in conjunction with the embodiments below, it will be understood that they are not intended to limit the integrated apparatus and method to these embodiments and examples. On the contrary, the integrated apparatus and method is intended to cover alternatives, modifications and equivalents, which may be included within the spirit and scope of the integrated apparatus and method as defined by the appended claims. Furthermore, in the following detailed description of the integrated apparatus and method, numerous specific details are set forth in order to more fully illustrate the integrated apparatus and method. However, it will be apparent to one of ordinary skill in the prior art that the integrated apparatus and method may be practiced without these specific details. In other instances, well-known methods and procedures, components and processes haven not been described in detail so as not to unnecessarily obscure aspects of the integrated apparatus and method.

FIG. 1 illustrates an exemplary block diagram of an integrated device according to an embodiment. An integrated microfluidic cartridge 10 includes a sample input chamber 12, a processing chamber 14, and an output chamber 18, coupled via microfluidic circuitry 24, 26. Microfluidic circuitry regulates fluid flow into, out of and through the cartridge 10, and specifically regulates flow of the sample within the cartridge 10. The microfluidic circuitry includes microfluidic pathways and valves. One or more solutions modules 30 are coupled to the cartridge 10. The cartridge 10 is also coupled to waste 32. For example, the solutions modules 30 are coupled to the sample input chamber 12 via microfluidic circuitry 20 and to the processing chamber 14 via microfluidic circuitry 22, and the processing chamber 14 is coupled to waste via microfluidic circuitry 28. In some embodiments, the solutions modules 30 and the waste 32 are separate from the cartridge 10. In other embodiments, the solutions modules 30 and/or the waste 32 are included as part of the cartridge 10. It is understood that the microfluidic circuitry 20, 22, 24, 26, 28 shown in FIG. 1 is for exemplary purposes only, and that alternative fluid pathways and distribution structures can be used. In general, microfluidic circuitry is used to supply and transport the various solutions, buffers, reagents, fluid sample mixtures, and the like between the various components within the cartridge 10, and to and from any external components in fluid communication with the cartridge 10. The microfluidic circuitry also includes individual valves and/or distribution valves to regulate the position of the various fluid elements within the cartridge 10. In some embodiments, the microfluidic circuitry includes a pump mechanism to enable fluid movement and to regulate fluid flow rate.

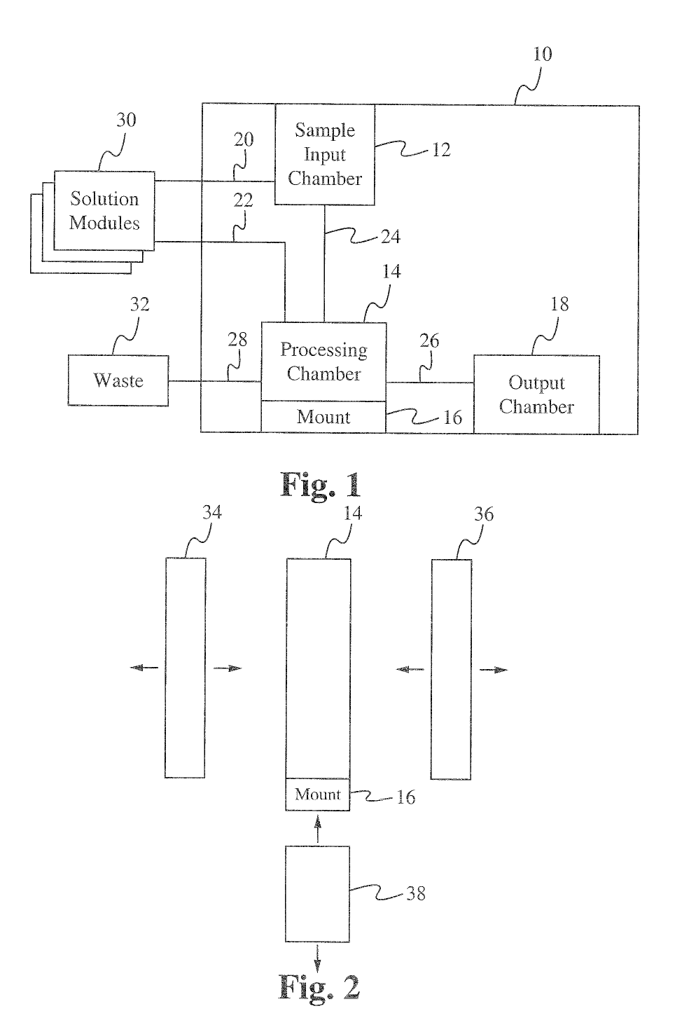

The processing chamber 14 is used to lyse one or more different cell types within a given input sample, to purify one or more nucleic acids from within the lysed cell types, and to amplify the one or more nucleic acids. FIG. 2 illustrates a side view of processing chamber 14. The cartridge 10 (FIG. 1) and the processing chamber 14 within the cartridge 10 are configured to enable coupling between the processing chamber 14 and a heat exchanger 34, a magnet 36, and a sonication device 38. In some embodiments, a mount 16 is coupled to the processing chamber 14. The mount 16 is configured to mate with the sonication device 38. In other embodiments, the sonication device 38 is coupled directly to the processing chamber 14.

In some embodiments, the sonication device 38 is configured to move between a first position and a second position. In the first position, the sonication device 38 contacts the mount 16. In the second position, the sonication device 38 is not in contact with the mount 16. In this manner, the sonication device 38 can be moved toward and away from the processing chamber 14. In other embodiments, the sonication device 38 is configured to be stationary. In this embodiment, the sonication device 38 is coupled to the mount 16.

The magnet 36 is configured to move between a first position and a second position. In the first position, the magnet 36 contacts a surface of the processing chamber 14. In the second position, the magnet 36 is not in contact with the processing chamber 14. In this manner, the magnet 36 can be moved toward and away from the processing chamber 14.

In an alternative configuration, the magnet 36 is not included in the system. Instead, a filter or other filtering means is implemented at each input and output port of the processing chamber 14. For example, a filter is positioned at each of the ports where the microfluidic circuitry 22, 24, 26, and 28 connects with the processing chamber 14. The filter, or other filtering means, is configured to retain beads within the processing chamber 14, as described in detail below.

The heat exchanger 34 is any conventional heating, cooling, or thermal cycling device that transfers heat to and/or from the processing chamber 14. In some embodiments, the heat exchanger 34 is configured to move between a first position and a second position. In the first position, the heat exchanger 34 contacts a surface of the processing chamber 14. In the second position, the heat exchanger 34 is not in contact with the processing chamber 14. In this manner, the heat exchanger 34 can be moved toward and away from the processing chamber 14. In other embodiments, the heat exchanger 34 is configured to be stationary. In this embodiment, the heat exchanger 34 is coupled to a surface of the processing chamber 14.

In some embodiments, the heat exchanger 34, the magnet 36, and the sonication device 38 are separate from the cartridge 10. For example, the cartridge 10 can be coupled to a mounting assembly that includes a heat exchanger, magnet, and sonication device that are configured to be coupled to the processing chamber 14. In other embodiments, the heat exchanger 34, the magnet 36, and/or the sonication device 38 are integrated as part of the cartridge.

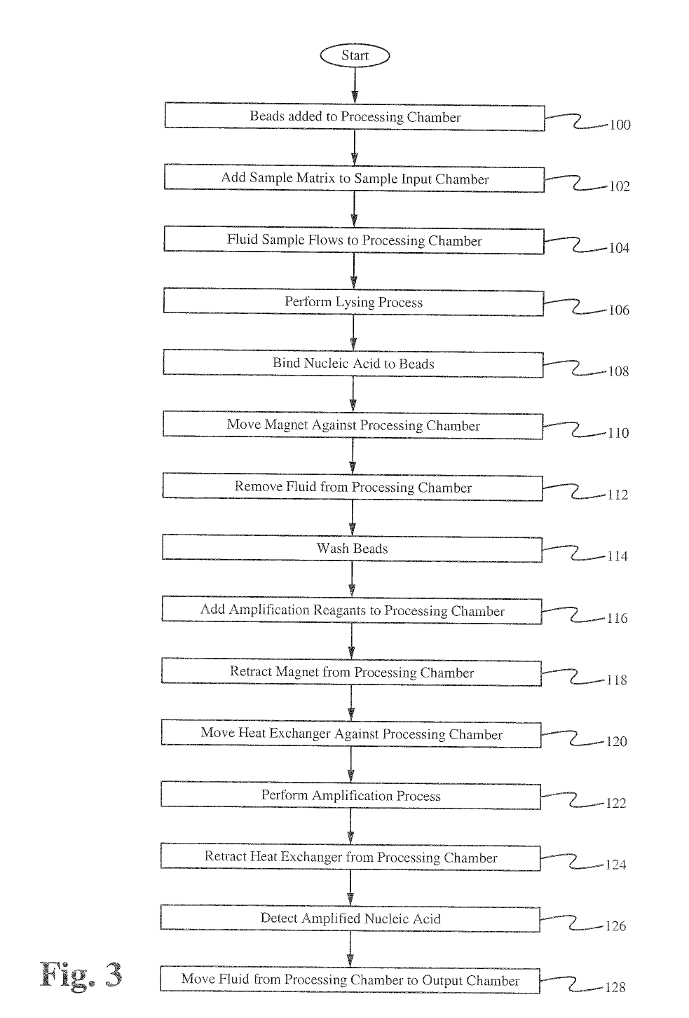

FIG. 3 illustrates a flow chart of the sample processing method according to an embodiment. The sample processing method is described relative to operation of the system of FIG. 1. It is understood that the sample processing method can be applied to alternative system and system component configurations.

At the step 100, beads are placed in the processing chamber 14. In some embodiments, the beads are silica beads. Nucleic acid is known to bind to the surface of silica beads. In other embodiments, each bead is coated with one or more capture moieties. Each capture moiety is configured to bind with a specific type of nucleic acid. In the case where the magnet 36 is used, the beads are paramagnetic. In the case where filters are used and not the magnet, then the beads are not paramagnetic. The beads can be placed in the processing chamber at the time the cartridge 10 is manufactured, or the beads can be added to the processing chamber post-production via microfluidic circuitry. The beads can be stored within a bead metering module, where the bead metering module is coupled to the processing chamber via microfluidic circuitry. Alternatively, one of the solutions modules 30 includes the beads and binding solution, which are added to the processing chamber 14.

At the step 102, a sample matrix including a sample is added to the sample input chamber 12. The sample matrix can be any matrix that includes a plurality of cells to be processed. Examples of the sample matrix include, but are not limited to, protopaper or cytobrush. In some embodiments, a lysis reagent and a binding buffer are added from the solutions module 30 to the sample input chamber 12 via microfluidic circuitry 20. In other embodiments, the sample input chamber is pre-loaded with the lysis reagent and the binding buffer. In still other embodiments, only a binding buffer is applied at this time. In still other embodiments, neither a lysis reagent nor a binding buffer are added at the step 102. The plurality of cells on the sample matrix bind to the binding buffer, forming a fluid sample including the plurality of cells. At the step 104, the fluid sample and the lysis reagent flow from the sample input chamber 12 into the processing chamber 14 via microfluidic circuitry 24. In some embodiments, if the lysis reagent has not already been added, then the lysis reagent is added at this time from the solutions module 30 to the processing chamber 14 via microfluidic circuitry 22. In other embodiments, a lysis reagent is not added at the step 104. At the step 106, a lysing process is then performed on the plurality of cells while in the processing chamber 12. In some embodiments, the lysing process is performed by sonication. In this case, the sonication device 38 is coupled to the mount 16, and the sonication energy provided by the sonication device 38 lyses the plurality of cells to release nucleic acid. In some embodiments, a lysis reagent, a binding buffer, or a lysis reagent and a binding buffer are added to the sample input chamber 12 after sonication is performed. In some embodiments, the lysing process is aided by applying heat to the processing chamber, the heat can be applied by thermally coupling the heat exchanger 34 to the processing chamber 14. During the lysing process in this embodiment, the heat exchanger 34 functions as a heater. The lysing process is performed for a period of time. The lysing process can be performed by applying both sonication and heat to the processing chamber 14.

As the resulting nucleic acid is released from the lysed cells, at the step 108 the nucleic acid binds to the beads, or the capture moiety coating the beads, positioned within the processing chamber 14. In some embodiments, sonication energy can be applied beyond the lysing process period of time so as to agitate the fluid and bead mixture, thereby improving exposure of the released nucleic acid to the beads. The binding process proceeds for an additional period of time.

Once the binding process is completed, at the step 110 the magnet 36 is moved to the first position against the surface of the processing chamber 14. The beads are paramagnetic and are attracted to the magnet 36 positioned against the processing chamber surface. The magnet 36 holds the beads within the processing chamber 14 while the fluid is removed from the processing chamber at the step 112. The fluid is removed via microfluidic circuitry 28 to waste 32 at the step 114. A wash solution is then added from the solutions modules 30 to the processing chamber 14. In some embodiments, the wash solution is retained in the processing chamber 14 for a period of time by closing the microfluidic circuitry 28, then removed via the microfluidic circuitry 28. During this wash period, the magnet 36 can either remain in the first position against the surface of the processing chamber 14, thereby holding the beads in place, or the magnet 36 can be retracted from the surface of the processing chamber 14 so that the beads are not held in place and allowed to mix with the wash solution. In either case, before the wash solution is removed, the magnet 36 is positioned against the surface of the processing chamber 14 so as to hold the beads in place.

In other embodiments, the microfluidic circuitry 28 remains open as the wash solution is added to the processing chamber 14 so that the wash solution flows through the processing chamber. In this case, the magnet 36 remains in the first position against the surface of the processing chamber 14, with the beads held in place, while the wash solution flows through the processing chamber 14. The wash solution may be retained for some period of time in the processing chamber 14 if the flow rate of the wash solution out of the processing chamber 14 is insufficient to match the flow rate of the wash solution into the processing chamber 14.

Once the wash solution is removed, amplification reagents are added to the processing chamber 14 at the step 116. In the case where amplification is to be performed using PCR, the amplification reagents are PCR reagents at the step 118. The magnet 36 is then retracted from the first position to the second position so that the magnet 36 is no longer in contact with the surface of the processing chamber 14, thereby releasing the beads to mix with the amplification reagents. At the step 120, the heat exchanger 34 is then moved to the first position against the surface of the processing chamber 14. At the step 122, nucleic acid bound to the beads within the processing chamber 14 are then amplified. In some embodiments, a thermal cycling process is used to perform the amplification process. The heat exchanger 34 cycles between various temperatures according to a defined temperature and time period schedule. For example, the heat exchanger 34 cycles between a first temperature for a first period of time and a second temperature for a second period of time, this cycling being performed for a predetermined number of cycles. Once the amplification process is completed, at the step 124 the heat exchanger 34 is retracted to the second position away from the surface of the processing chamber 14.

In some embodiments, the cartridge 10 and the processing chamber 14 are configured such that the interior of the processing chamber 14, or a portion thereof, is optically accessible to detect the presence of the amplified nucleic acid while still present within the processing chamber. In some embodiments, the processing chamber 14 and the corresponding external structure of the cartridge 10 are made of an optically transparent material. In some configurations, the surface of the processing chamber 14 is also the outer surface of the cartridge 10 at the processing chamber 14. The nucleic acid present within the processing chamber 14 includes the nucleic acid still bound to the beads, and also the amplified nucleic acid floating within the fluid. At the step 126, detection of the amplified nucleic acid within the processing chamber 14 can be performed in real-time or at the end of the amplification process. Optical detection of the amplified nucleic acid is well known, and can include detection of fluorescent dyes released during the amplification process, such as the use of Taqman® probes.

At the step 128, the fluid including the amplified nucleic acid is removed from the processing chamber 14 via the microfluidic circuitry 26 and input to the output chamber 18. In some embodiments, the beads are retained in the processing chamber 14 while the fluid is removed. The beads are retained using the magnet 36. In other embodiments, the fluid and the beads are removed from the processing chamber 14 and input to the output chamber 18. Contents of the output chamber 18 can then be removed for external analysis.

In the case where filters are used instead of the magnet to retain the beads within the processing chamber, the steps related to moving the magnet into proper position are not necessary. Instead, as the fluid is removed from the processing chamber, such as the removal of the lysing reagents and the lysate, the filters allow the fluid to exit while the beads are retained in the processing chamber. The filters perform a similar function during the removal of the wash solution.

In some embodiments, the cartridge 10 is designed as a disposable unit so that once the contents of the output chamber are removed, or after detection of the amplified nucleic acids within the processing chamber 14 is performed, the cartridge 10 is discarded to waste.

The integrated apparatus and method combines both the sample preparation and the amplification process on beads within a single processing chamber. Sample preparation is performed on beads followed by amplification either by 3′-5′ exonuclease Taqman® or by any number of nucleic acid amplification methods including, but not exclusive of, polymerase chain reaction. Nucleic acid captured on the beads is not eluted for amplification, instead amplification of the captured nucleic acid is performed while the nucleic acid is still bound to the beads. Real-time or end-point detection can be performed within the processing chamber, or the amplified product can be removed for subsequent analysis.

Sample preparation on paramagnetic or silica beads followed by amplification of the nucleic acid signature on the same media integrates the sample preparation and analysis methods into a “one pot analysis” from start to finish. For example, such a one pot analysis can be used for analysis of a biological sample in a single-use disposable unit, such as the cartridge 10. In this instance, a clinical sample represented on a cytobrush, for example, is introduced into a self-contained unit that has the capability of introducing the reagents required for the sample clean up, nucleic acid attachment to the beads, and amplification. Exposing nucleic acid from the sample, such as during the lysing process, also releases other substances into the lysed matrix that may inhibit or compromises subsequent reactions during downstream processing and analysis. Sample clean up refers to removing these substances while leaving the nucleic acid behind for subsequent processing and analysis. Performing sample clean up makes the detection system robust and reliable from sample to sample, and it also makes the detection system independent on the type of sample matrix. For example, the sample matrix can be a clinical sample that has, for example, blood, urine, or feces, or the sample matrix can be an environmental sample that has, for example, humic substances, pesticides, etc. Thus, the sample is introduced into one site, the self-contained unit, to which the reagents are delivered from the start to finish of the analysis. This approach prevents dilution loss as well as other fluidic challenges that arise from having to physically move the sample from one port to another. Alternatively, the apparatus and method can be implemented in a non-integrated manner, where some or all of the components are separate from each other, and some or all of the components are reusable.

In an exemplary application, the sample preparation process is performed using the PrepSeeq Sample Preparation® kit from Life Technologies®. Control experiments are set up with sample preparation using the protocol suggested by the manufacturer. Amplification on the beads is performed using the sample preparation protocol, but the amount of beads called for in the protocol is reduced. In one application, approximately 1/100th the volume of beads called for in the PrepSeeq Sample Preparation® protocol is used. When the full volume of beads suggested by the manufacturer is used, the reaction is completely inhibited. The amplification is done with both the construct of the Human Pappiloma Virus (HPV) and the Bacillus anthracis nucleic acid.

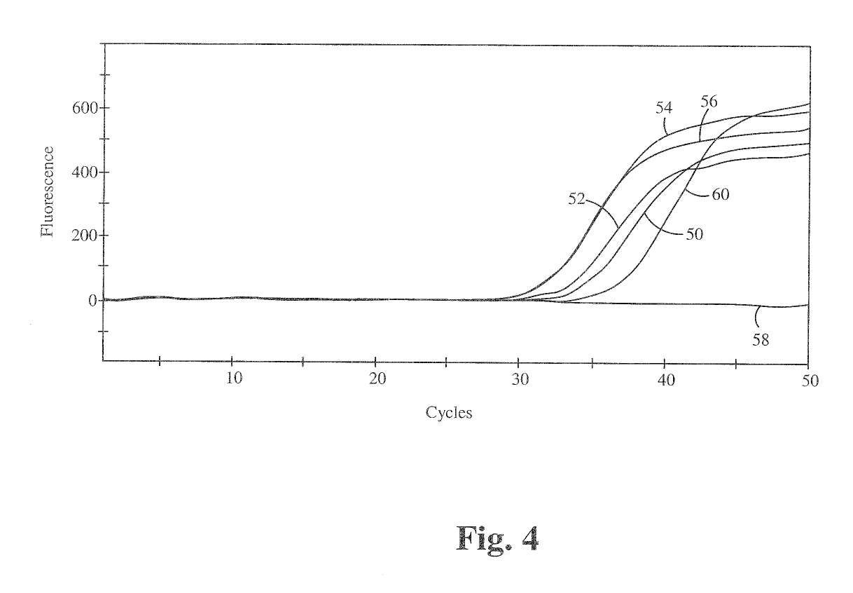

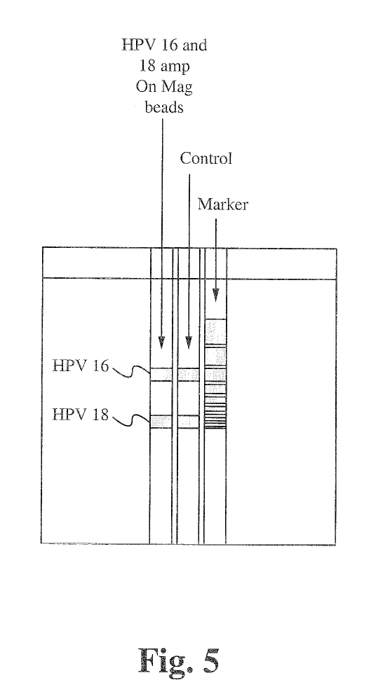

FIG. 4 illustrates an exemplary graph of the amplification versus number of cycles for the sample preparation and amplification process applied to a Bacillus anthracis nucleic acid. Curves 50 and 52 represent the amplification carried out in real-time using Taqman® reaction of two different samples of Bacillus anthracis nucleic acid, where amplification and sample preparation are performed on the beads. Curves 54 and 56 represent the amplification for two positive controls. Each positive control indicates that the amplification process is working correctly. Curve 58 represents the amplification for a negative control. In the negative control sample, no nucleic acid is present. The negative control indicates that there is no contamination. Curve 60 represents the amplification for another sample of the Bacillus anthracis nucleic acid, where sample preparation is performed on the beads, but the captured nucleic acid is elated from the beads and the eluate is subjected to the amplification process.

FIG. 5 illustrates an exemplary gel electrophoresis analysis performed on an amplified Human Pappiloma Virus (HPV). The amplified sample was processed by performing the sample preparation and amplification on the beads. The HPV sample was purified and amplified in a duplex, HPV 16 and HPV 18, on the beads. The left column represents the duplex amplification of the HPV 16 and the HPV 18 nucleic acids on the beads. The top band in the left column corresponds to the HPV 16 nucleic acid, and the bottom band in the left column correspond to the HPV 18 nucleic acid. The middle column represents a control value for the HPV 16 nucleic acid and the HPV 18 nucleic acid. The control values represent the amplification profile obtained when the HPV 16 nucleic acid and the HPV 18 nucleic acid are conventionally prepared and amplified without the use of beads. The right column represents a nucleic acid size marker.

The integrated apparatus and method has been described in terms of specific embodiments incorporating details to facilitate the understanding of the principles of construction and operation of the integrated apparatus and method. The specific configurations shown and the methodologies described in relation to the various modules and the interconnections therebetween are for exemplary purposes only. Such reference herein to specific embodiments and details thereof is not intended to limit the scope of the claims appended hereto. It will be apparent to those skilled in the art that modifications may be made in the embodiment chosen for illustration without departing from the spirit and scope of the integrated apparatus and method.

What is claimed is:

1. A method of processing a sample, the method comprising:
a. providing a processing vessel including a plurality of beads, each bead is configured to bind with nucleic acid;
b. adding a fluid sample and lysing reagents to the processing vessel, the fluid sample including one or more different types of cells;
c. lysing the cells within the processing chamber, thereby releasing nucleic acid from within the cells, wherein the nucleic acid binds to the plurality of beads;
d. retaining the plurality of beads within the processing chamber while removing the fluid sample and lysing reagents;
e. adding amplification reagents to the processing vessel; and
f. performing an amplification process within the processing vessel, wherein the nucleic acid bound to the plurality of beads is amplified.

2. The method of claim 1 wherein performing the amplification process yields an amplified product including the nucleic acid bound to the beads and nucleic acid in solution.

3. The method of claim 1 further comprising detecting the amplified nucleic acid within the processing vessel.

4. The method of claim 3 wherein the amplified nucleic acid is detected in real-time or at the end of the amplification process.

5. The method of claim 1 wherein the amplification process comprises polymerase chain reaction.

6. The method of claim 1 wherein lysing the cells comprises sonicating the cells.

7. The method of claim 1 wherein lysing the cells comprises applying heat to the processing vessel.

8. The method of claim 1 wherein each bead is a paramagnetic bead, and retaining the plurality of beads comprises applying a magnet to the processing vessel.

9. The method of claim 1 wherein each bead is a silica bead, and retaining the plurality of beads comprises filtering the beads as the fluid sample and lysing reagents are removed from the processing vessel.

10. The method of claim 1 further comprising adding a wash buffer to the processing chamber after removing the fluid sample and lysing reagents, and retaining the plurality of beads within the processing chamber while removing the wash buffer.

11. The method of claim 1 wherein each bead is coated with one or more capture moieties, each capture moiety configured to bind with a specific type of nucleic acid, further wherein released nucleic acid of the specific types binds to the plurality of beads.

12. An apparatus for processing a sample, the apparatus comprising:
a. a sample input chamber configured to receive as input a sample including a plurality of cells; and
b. a processing chamber including a plurality of beads positioned therein, each bead is configured to bind with nucleic acid, wherein the processing chamber is coupled to the sample input chamber to receive a fluid sample that includes the plurality of cells, further wherein the processing chamber is configured to receive lysing reagents and to lyse the plurality of cells, thereby releasing nucleic acid that binds to the plurality of beads, to remove the fluid sample and lysing reagents while retaining the plurality of beads, to receive amplification reagents, and to perform an amplification process thereby amplifying the nucleic acid bound to the plurality of beads.

13. The apparatus of claim 12 wherein at least a portion of the processing chamber is transparent or provides optical access to the interior to detect the amplified nucleic acid.

14. The apparatus of claim 12 wherein the processing chamber includes a mounting seat configured to be removably coupled to a sonication device, further wherein the processing chamber and the sonication device are configured to lyse the plurality of cells using sonication.

15. The apparatus of claim 12 wherein the processing chamber includes a first surface configured to be removably coupled to a heat exchanger, further wherein the processing chamber and the heat exchanger are configured to thermally cycle contents of the processing chamber, the thermal cycling used to perform the amplification process.

16. The apparatus of claim 15 wherein the processing chamber and the heat exchanger are further configured to lyse the plurality of cells by heating the contents of the processing chamber.

17. The apparatus of claim 12 wherein the processing chamber includes a second surface configured to be removably coupled to a magnet, further wherein each bead is paramagnetic, and the processing chamber and the magnet are configured to retain the plurality of beads within the processing chamber while the fluid sample and lysing reagents are removed.

18. The apparatus of claim 12 wherein the processing chamber includes a filter coupled to each fluid input port and to each fluid output port to retain the plurality of beads within the processing chamber while the fluid sample and lysing reagents are removed.

19. The apparatus of claim 12 further comprising one or more solutions modules coupled to the processing chamber to provide the lysing reagents and the amplification reagents.

20. The apparatus of claim 12 wherein each bead is coated with one or more capture moieties, each capture moiety configured to bind with a specific type of nucleic acid, further wherein released nucleic acid of the specific types binds to the plurality of beads.

21. The apparatus of claim 12 further comprising microfluidic circuitry coupled to the sample input chamber and to the processing chamber, the microfluidic circuitry configured to regulate fluid flow between the sample input chamber and the processing chamber, and to regulate fluid flow into and out of the processing chamber.

22. The apparatus of claim 12 wherein the apparatus comprises an integrated cartridge.

23. A system for processing a sample, the system comprising:
a. a sample input chamber configured to receive as input a sample including a plurality of cells;
b. a processing chamber including a plurality of beads positioned therein, each bead is configured to bind with nucleic acid, wherein the processing chamber is coupled to the sample input chamber to receive a fluid sample that includes the plurality of cells;
c. lysing means coupled to the processing chamber to lyse the plurality of cells within the processing chamber, thereby releasing nucleic acid that binds to the plurality of beads;
d. retention means coupled to the processing chamber to retain the plurality of beads within the processing chamber while fluid is removed from the processing chamber; and
e. amplification means coupled to the processing chamber to amplify the nucleic acid bound to the plurality of beads.

24. The system of claim 23 wherein the lysing means comprises a sonication device.

25. The system of claim 23 wherein the lysing means and the amplification means comprise a heat exchanger.

26. The system of claim 23 further comprising microfluidic circuitry coupled to the sample input chamber and to the processing chamber, the microfluidic circuitry configured to regulate fluid flow between the sample input chamber and the processing chamber, and to regulate fluid flow into and out of the processing chamber.

27. The system of claim 23 further comprising one or more solutions modules coupled to the processing chamber to provide lysing reagents, amplification reagents, and buffer solutions.

28. The system of claim 23 wherein each bead is coated with one or more capture moieties, each capture moiety configured to bind with a specific type of nucleic acid, further wherein released nucleic acid of the specific types binds to the plurality of beads.

29. The system of claim 23 wherein the retention means comprises a filter coupled to each fluid input port and to each fluid output port of the processing chamber, the filters are configured to retain the plurality of beads within the processing chamber while fluid removed from the processing chamber.

30. The system of claim 23 wherein the retention means comprises a magnet configured to be removably coupled to the processing chamber, further wherein each bead is a paramagnetic bead, and the processing chamber and the magnet are configured to retain the plurality of beads within the processing chamber while fluid removed from the processing chamber.
